(19) United States
(12) Patent Application Publication
(10) Pub. No.: US 2023/0330151 A1
(43) Pub. Date: Oct. 19, 2023

(54) COMPOSITION FOR PROMOTING DIFFERENTIATION OF NEURAL STEM CELLS INTO DOPAMINERGIC NEURONS

(71) Applicants: CHA UNIVERSITY INDUSTRY-ACADEMIC COOPERATION FOUNDATION, Pocheon-si (KR); SUNGKWANG MEDICAL FOUNDATION, Seoul (KR)

(72) Inventors: Jisook MOON, Seoul (KR); Chul KIM, Anyang-si (KR); Jae Hyun PARK, Wonju-si (KR); Ji-Min PARK, Gwangju-si (KR); Young Sook SONG, Seoul (KR); Chul Woo LIM, Seoul (KR); Yuri CHOI, Seoul (KR)

(73) Assignees: CHA UNIVERSITY INDUSTRY-ACADEMIC COOPERATION FOUNDATION, Pocheon-si (KR); SUNGKWANG MEDICAL FOUNDATION, Seoul (KR)

(21) Appl. No.: 18/042,635

(22) PCT Filed: Aug. 24, 2021
(86) PCT No.: PCT/KR2021/011263

§ 371 Date: Feb. 23, 2023

(30) Foreign Application Priority Data
Aug. 24, 2020 (KR) 10-2020-0106224
Aug. 24, 2021 (KR) 10-2021-0111440

(51) Int. Cl. A61K 35/30; C12N 5/0793

(52) U.S. Cl. CPC A61K 35/30; C12N 5/0619; C12N 2500/40; C12N 2500/38; C12N 2500/74

(57) ABSTRACT

Provided are a composition and a method for promoting differentiation of neural stem cells into dopaminergic neurons, the composition including fusaric acid, ascorbic acid, nicotinamide adenine dinucleotide, or a combination thereof. The composition and method according to an aspect may not only increase differentiation of neural stem cells isolated at an early stage of development into dopaminergic neurons, but also increase differentiation of subcultured neural stem cells into dopaminergic neurons, and thus, it is possible to secure more dopaminergic neurons, and increase therapeutic effects on Parkinson's disease.

### TECHNICAL FIELD

This application claims priority to Korean Patent Application No. 10-2020-0106224, filed on Aug. 24, 2020, and Korean Patent Application No. 10-2021-0111440, filed on Aug. 24, 2021, the disclosures of which are incorporated herein by reference in their entirety.

The present application relates to a composition and a method for promoting differentiation of neural stem cells into dopaminergic neurons, the composition including fusaric acid, ascorbic acid, nicotinamide adenine dinucleotide, or a combination thereof.

### BACKGROUND ART

As the population is aging rapidly, the number of patients with degenerative neurological diseases is rapidly increasing. Parkinson's disease, one of the degenerative diseases of the nervous system, is a disease characterized by selective degeneration of dopaminergic neurons in the substantia nigra region of the midbrain, and a treatment method in which dopaminergic neurons in patients with Parkinson's disease are replaced through transplantation of fetal midbrain tissue has been studied in various ways. However, although transplantation treatment may be effective for the treatment of Parkinson's disease, since it is difficult to obtain a large amount of human aborted fetal tissues, its clinical application is limited to very few cases. To overcome such issues, various cells have been studied as donor cell candidates for transplantation treatment of Parkinson's disease. In addition, dopaminergic functions are involved in various diseases such as schizophrenia, autism, attention deficit hyperactivity disorder, and drug abuse. Dopamine is deeply related to reward seeking behaviors such as consumption and addiction.

Among various donor cell candidates, human neural progenitor cells (hNPCs) derived from fetal midbrain tissue have long-term proliferative activity, and thus have an excellent self-proliferation ability, and are capable of differentiating into dopaminergic neurons, and therefore, are expected to be useful as a cell source. Therefore, it is very important to establish a method of more efficiently proliferating (or expanding) hNPCs, as well as a method of effectively differentiating hNPCs into dopaminergic neurons, for the treatment of Parkinson's disease and various dopamine-related diseases through substitution, that is, transplantation of dopaminergic neurons.

As a method of differentiating hNPCs into dopaminergic neurons, a method of differentiating hNPCs for 3 weeks by adding brain-derived neurotrophic factor (BDNF), dopamine, and forskolin to the medium (Riaz, S. S. et al., Brain Res. See Dev. Brain Res. 2004; 153(1), 39-51); and methods using sonic hedgehog (SHH), fibroblast growth factor-8 (FGF-8), brain-derived neurotrophic factor (BDNF), etc. are known, but differentiation methods in the art have unsatisfactory differentiation efficiency, require a long time for differentiation, and have economic difficulties due to the use of a medium containing a large amount of expensive additives such as SHH or FGF-8. In addition, there is a limitation in clinical use due to a lack of technology for proliferating a large number of cells necessary for the treatment of patients with Parkinson's disease.

With this background, it was confirmed that dopaminergic neurons may be more efficiently proliferated from neural stem cells by using fusaric acid, ascorbic acid, nicotinamide adenine dinucleotide (NAD+), or a combination thereof, and thus, the present disclosure was completed.

### DISCLOSURE

### Technical Problem

Provided is a composition for promoting differentiation of neural stem cells into dopaminergic neurons, including: fusaric acid, ascorbic acid, nicotinamide adenine dinucleotide (NAD+), or a combination thereof.

Another aspect provides a method of promoting differentiation of neural stem cells into dopaminergic neurons, including culturing neural stem cells in a medium including fusaric acid, ascorbic acid, nicotinamide adenine dinucleotide, or a combination thereof.

Still another aspect provides dopaminergic neurons differentiated by the method of promoting differentiation of neural stem cells into dopaminergic neurons.

Still another aspect provides a pharmaceutical composition for preventing or treating Parkinson's disease including neural stem cells and the composition for promoting differentiation of the neural stem cells into dopaminergic neurons, as active ingredients.

Still another aspect provides a method of preventing or treating Parkinson's disease, including administering the pharmaceutical composition to a subject.

Still another aspect provides use of the composition for promoting differentiation of neural stem cells into dopaminergic neurons for preparing a drug for preventing or treating Parkinson's disease.

### Technical Solution

An aspect provides a composition for promoting differentiation of neural stem cells into dopaminergic neurons, including fusaric acid, ascorbic acid, nicotinamide adenine dinucleotide, or a combination thereof.

The composition may promote differentiation potency of stem cells. The term “differentiation potency”, used herein, refers to an ability of stem cells to specialize their structure or function to differentiate into adipocytes, osteoblasts, chondroblasts, myofibroblasts, muscle cells, nerve cells, etc., and differentiation potency used herein may refer to differentiation potency of stem cells to differentiate into dopaminergic neurons.

In this specification, the term “fusaric acid (FA)” refers to a picolinic acid derivative, which may also be called 5-butylpicolinic acid, fusaric acid, or fusarinic acid. Fusaric acid is an antibiotic first isolated from Fusarium heterosporium.

The fusaric acid may be included in an amount of 10 μM to 500 μM, for example, 10 μM to 490 μM, for example, 20 μM to 480 μM, for example, 20 μM to 470 μM, for example, 30 μM to 460 μM, for example, 30 μM to 450 μM, for example, 40 μM to 440, for example, 40 μM to 430 μM, for example, 50 μM to 420 μM, for example, 50 μM to 410 μM, for example, 50 μM to 400 μM, for example, 50 μM to 350 μM, for example, 50 μM to 300 μM, for example, 50 μM to 250 μM, for example, 50 μM to 200 μM, for example, 50 μM to 150 μM.

In an embodiment, when the fusaric acid is included in the composition in an amount of 10 μM to 500 μM, specifically 50 μM to 150 μM, differentiation of neural stem cells into dopaminergic neurons may be promoted. In an embodiment, when neural stem cells that have been subcultured 10 or more times and 30 or less times are differentiated in a composition including 50 μM to 150 μM of fusaric acid, differentiation into dopaminergic neurons may be promoted.

The term “ascorbic acid (AA)”, used herein, refers to one of water-soluble vitamins which is an organic compound having antioxidant properties. It is also called vitamin C, and ascorbic acid deficiency is known to cause scurvy.

The ascorbic acid may be included in an amount of 10 μM to 500 μM, for example, 10 μM to 490 μM, for example, 20 μM to 480 μM, for example, 20 μM to 470 μM, for example, 30 μM to 460 μM, for example, 30 μM to 450 μM, for example, 40 μM to 440, for example, 40 μM to 430 μM, for example, 50 μM to 420 μM, for example, 50 μM to 410 μM, for example, 50 μM to 400 μM, for example, 50 μM to 350 μM, for example, 50 μM to 300 μM, for example, 50 μM to 250 μM, for example, 50 μM to 200 μM, for example, 50 μM to 150 μM.

In an embodiment, when the ascorbic acid is included in the composition in an amount of 10 μM to 500 μM, specifically 50 μM to 150 μM, differentiation of neural stem cells into dopaminergic neurons may be promoted. In an embodiment, when neural stem cells that have been subcultured 10 or more times and 30 or less times are differentiated in a composition including 50 μM to 150 μM of ascorbic acid, differentiation into dopaminergic neurons may be promoted.

In this specification, the term “nicotinamide adenine dinucleotide (NAD+)” refers to a coenzyme involved in several oxidoreductases, and is also called diphosphopyridine nucleotide (DPN) or coenzyme I (Co I). According to oxidation-reduction of the substrate, the nicotinamide moiety of NAD is reduced and oxidized to act as a coenzyme. It is known that most of NAD in living cells exists in an oxidized form and is involved in oxidative degradation of organic compounds.

The NAD+ may be included in an amount of 0.1 mM to 4 mM, for example, 0.1 mM to 3.8 mM, for example, 0.1 mM to 3.6 mM, for example, 0.1 mM to 3.4 mM, for example, 0.1 mM to 3.2 mM, for example, 0.1 mM to 3 mM, for example, 0.1 mM to 2.8 mM, for example, 0.1 mM to 2.6 mM, for example, 0.1 mM to 2.4 mM, for example, 0.1 mM to 2.2 mM, for example, 0.1 mM to 2 mM, for example, 0.1 mM to 1.5 mM, for example, 0.1 mM to 1 mM, for example, 0.2 mM to 2 mM, for example, 0.2 mM to 1.5 mM, for example, 0.4 mM to 2 mM, for example, 0.4 mM to 1.5 mM.

In an embodiment, when NAD+ is included in the composition in an amount of 0.1 mM to 4 mM, specifically 0.4 mM to 1.5 mM, differentiation of neural stem cells into dopaminergic neurons may be promoted. In an embodiment, when neural stem cells that have been subcultured 10 or more times and 30 or less times are differentiated in a composition including 0.4 mM to 1.5 mM of NAD+, differentiation into dopaminergic neurons may be promoted.

The composition may include a combination of fusaric acid and ascorbic acid, a combination of fusaric acid and nicotinamide adenine dinucleotide, a combination of ascorbic acid and nicotinamide adenine dinucleotide, or a combination of fusaric acid, ascorbic acid and nicotinamide adenine dinucleotide.

The composition may include fusaric acid, ascorbic acid, and nicotinamide adenine dinucleotide in a concentration ratio (μM) of 1 to 5:1 to 10:5 to 100. The composition may remarkably increase differentiation efficiency of neural stem cells into dopaminergic neurons, by including fusaric acid, ascorbic acid, and nicotinamide adenine dinucleotide in the above concentration range.

In an embodiment, the neural stem cells may be subcultured neural stem cells.

The term “subculture”, used herein, refers to a method of continuously culturing cells, specifically stem cells, in a healthy state for a long period of time, and may mean replacing a culture vessel or culturing a cell population in divisions. One-time replacement of the culture vessel or dividing and culturing the cell population is called subculture 1. The term “subculture” may be used interchangeably with “generation”.

The term “early stage” subculture, used herein, refers to when subculture is performed 1 or more times and less than 10 times, “middle stage” subculture refers to when subculture is performed 10 or more times and less than 20 times, and “late stage” subculture refers to when subculture is performed 20 times or more.

In an embodiment, the neural stem cells that have undergone subculture may have been subcultured 10 or more times and 30 or less times, 10 or more times and 25 or less times, 20 times or more and 30 or less times, or 10 or more times and less than 20 times.

In an embodiment, compared to a case where FA is added alone to differentiation medium of neural stem cells, when a combination of FA and AA, a combination of FA and NAD+, in particular, a combination of FA, AA, and NAD+ is added, efficiency of differentiation of neural stem cells into dopaminergic neurons is significantly increased, and more dopaminergic neurons can be secured.

In an embodiment, when a combination of FA, AA, and NAD+ is added to a differentiation medium of neural stem cells, efficiency of differentiation of neural stem cells, which were subcultured 10 or more times and 30 or less times, into dopaminergic neurons is significantly increased. For example, efficiency of differentiation of neural stem cells, which are subcultured 10 or more times and 20 or less times, into dopaminergic neurons is remarkably increased. Therefore, it was confirmed that a differentiation rate, which gradually decreases when subculture is performed 10 or more times, of neural stem cells into dopaminergic cells may be maintained or enhanced by culturing the neural stem cells in a differentiation medium supplemented with a combination of FA, AA, and NAD+.

The term “neural stem cell”, used herein, refers to a cell that has a self-renewal ability of continuously proliferating in an undifferentiated state, and has multipotency of differentiation of differentiating from one stem cell into various neurons and glia, and may be derived from an animal. In this regard, the animal includes not only humans and primates, but also animals such as cows, pigs, sheep, horses, dogs, mice, rats, and cats, and is preferably a human. In some cases, “neural stem cell” is also used to encompass a meaning of “neural progenitor cell”.

In the present specification, the “neural progenitor cells” may be used in the same sense as “progenitors”, “precursors”, and “precursor cells”.

In an embodiment, the neural stem cells may be embryonic stem cells, embryonic germ cells, embryonic carcinoma cells, induced pluripotent stem cells (iPSCs), or adult stem cells. In an embodiment, the neural stem cells may be embryonic stem cells isolated from the central nervous system of a fetus.

The term “neural cells”, used herein, refers to cells constituting the nervous system, and may be used in the same sense as “neurons”. The term “dopaminergic neural cells”, used herein, refers to nerve cells secreting dopamine, a neurotransmitter, and refers to nerve cells expressing tyrosine hydroxylases (THs). The term may be used interchangeably with “dopaminergic neurons”, “dopamine neurons”, “DA” and the like. It is known that dopaminergic neurons are specifically located in the substantia nigra of the midbrain and regulate postural reflexes, movement, and reward-related behaviors by stimulating the striatum, limbic system, and neocortex in vivo.

In an example, the dopaminergic neurons may be dopaminergic neural progenitors, or dopaminergic neural precursor cells, or mature dopaminergic neurons, but are not limited thereto.

In an example, the dopaminergic neurons may be midbrain dopaminergic neurons. The the term “midbrain dopaminergic neurons” means dopaminergic neurons observed in the midbrain region, for example, dopaminergic neurons observed in the ventral region of the midbrain, but is not limited thereto.

The term “differentiation”, used herein, means that cells develop into specific cells, and specifically, refers to a phenomenon in which a structure or function of a cell is specialized during its growth by dividing and proliferating, and refers to a change in a form or function for performing a given task. “Differentiation” of a neural stem cell is preceded by asymmetric division, in which a parent cell divides into two cells with different characteristics, some of the divided cells remain as stem cells identical to the parent cell, and some differentiate into specific cells. In that differentiation of neural stem cells is accompanied by such an asymmetric division process, “differentiation of neural stem cells” may include a meaning of “proliferation”. The term “proliferation”, used herein, refers to a phenomenon in which cells divide and proliferate, and specifically refers to a phenomenon in which cells of the same type are multiplied by division, that is, a case in which cells of the same type are reproduced and their number increases.

Another aspect provides a method of differentiating neural stem cells into dopaminergic neurons, including: subculturing neural stem cells; and differentiating the neural stem cells in a medium including fusaric acid, ascorbic acid, nicotinamide adenine dinucleotide, or a combination thereof. The fusaric acid, ascorbic acid, nicotinamide adenine dinucleotide, neural stem cells, dopaminergic neurons, differentiation, or subculture are as described above.

The subculturing may be performed under conditions used in a culturing method in the art. For example, the subculture may be performed at about 37° C. for 7 days to 14 days, preferably for about 7 days. In addition, the subculture may be performed under a hypoxia condition, for example, a hypoxia condition of oxygen partial pressure of 2% to 10%.

The differentiating may be performed under conditions used in a differentiating method in the art. For example, the differentiation may be performed at about 37° C. for 7 days to 14 days, preferably for about 7 days. In addition, the subculture may be performed under a hypoxia condition, for example, a hypoxia condition of oxygen partial pressure of 2% to 10%.

The term “medium”, used herein, refers to a medium capable of supporting growth, survival, and differentiation of stem cells in vitro, and includes all media in the art appropriate for culturing or differentiating stem cells. Depending on a cell type, a type of medium and culture conditions may be selected at a technical level in the related art. The medium used for culturing is specifically a cell culture minimum medium (CCMM), and may generally include a carbon source, a nitrogen source, and trace elements. The cell culture minimum medium may include, for example, Dulbecco's modified eagle's medium (DMEM), minimal essential medium (MEM), basal medium eagle (BME), RPMI1640, F-10, F-12, α-minimal essential medium (α-MEM), Glasgow's minimal essential medium (GMEM), Iscove's modified Dulbecco's medium, etc., but is not limited thereto. In addition, the medium may contain antibiotics such as penicillin, streptomycin, gentamicin, or a mixture of two or more thereof.

The differentiation medium of the neural stem cells may include fusaric acid, ascorbic acid, nicotinamide adenine dinucleotide, or a combination thereof for promoting differentiation of neural stem cells into dopaminergic neurons, without particular limitations in a medium type and a method of culturing. In addition to the fusaric acid, ascorbic acid, and nicotinamide adenine dinucleotide, one or more previously known culture and differentiation inducers may be used together. For example, the neural stem cell culture medium may further include B27-CTS, B27 supplement, forskolin, dibutyryl cAMP, and L-glutamine.

Still another aspect provides a pharmaceutical composition for preventing or treating Parkinson's disease including, as active ingredients, dopaminergic neurons differentiated by the method of promoting differentiation of the neural stem cells into dopaminergic neurons. The neural stem cells and dopaminergic neurons are as described above.

In an example, provided is a pharmaceutical composition for preventing or treating Parkinson's disease including, as active ingredients, the dopaminergic neurons differentiated from the pharmaceutical composition including neural stem cells and a composition for differentiation of the neural stem cells into dopaminergic neurons. The composition for differentiation of the neural stem cells into dopaminergic neurons, and neural stem cells are as described above.

The term, “Parkinson's disease”, used herein, refers to a degenerative brain disease of the nervous system caused by a loss of dopaminergic neurons. Resting tremor, stiffness, bradykinesia (slowness of movement), and postural instability are characteristic features, and it is known that clinical symptoms generally begin to appear after an age of 60.

The term, “prevention”, used herein, refers to all acts of suppressing or delaying progression of Parkinson's disease by administration of neural stem cells and the composition for promoting differentiation of the neural stem cells into dopaminergic neurons.

The term, “treatment”, used herein, refers to all acts of improving or beneficially altering Parkinson's disease by administration of neural stem cells and the composition for promoting differentiation of the neural stem cells into dopaminergic neurons.

The pharmaceutical composition may further include a pharmaceutically acceptable carrier, excipient, or diluent commonly used in the art to prepare a pharmaceutical composition, and the carrier may include a non-naturally occurring carrier. The “pharmaceutically acceptable” means exhibiting properties that are not toxic to cells or humans exposed to the composition. Specifically, a type of the carrier is not particularly limited, and any carrier commonly used and pharmaceutically acceptable in the art may be used. Non-limiting examples of the carrier include saline, sterile water, Ringer's solution, buffered saline, albumin injection solution, dextrose solution, maltodextrin solution, glycerol, ethanol, and the like. These may be used alone or in combination of two or more. In addition, when necessary, other additives in the art such as antioxidants, buffers, and/or bacteriostatic agents may be added and used, and diluents, dispersants, surfactants, binders, lubricants, etc. may be additionally added to formulate into injectable formulations such as aqueous solutions, suspensions, and emulsions, pills, capsules, granules, or tablets.

An administration method of the pharmaceutical composition for preventing or treating Parkinson's disease is not particularly limited, and may be according to a method commonly used in the art. In addition, the composition for preventing or treating Parkinson's disease may be formulated in various formulations according to an intended administration method.

Still another aspect provides a method of preventing or treating Parkinson's disease, including administering the pharmaceutical composition to a subject. The pharmaceutical composition, Parkinson's disease, prevention, and treatment are as described above.

The term “administration”, used herein, means introducing a given substance into a subject in an appropriate way.

The term “subject”, used herein, means all animals including humans, such as rats, mice, livestock, etc., which have or may have Parkinson's disease. Specifically, the subject may be a mammal including human. More specifically, the subject may include a companion animal. The “companion animal” refers to animals that live together with humans, and specific types include mammals such as dogs, cats, hamsters and guinea pigs, birds such as parrots and canaries, but is not limited thereto.

Specifically, the method of preventing or treating Parkinson's disease may include administering to a subject the pharmaceutical composition including a composition for promoting differentiation of neural stem cells into dopaminergic neurons or neural stem cells, in a pharmaceutically effective amount. The “pharmaceutically effective amount” means an amount that is sufficient to treat a disease at a reasonable benefit/risk ratio applicable to a medical treatment and does not cause side effects, and the effective amount may be readily determined by one skilled in the art according to factors including a patient's sex, age, body weight, and health condition, a type and severity of the disease, an activity of a drug, sensitivity to a drug, a method of administration, time of administration, a route of administration, and a rate of excretion, duration of treatment, drugs used in combination or concurrently, and other factors well known in the medical field.

The pharmaceutical composition may be administered as an individual therapeutic agent or in combination with other therapeutic agents, or may be administered sequentially or concurrently with other therapeutic agents. In addition, the pharmaceutical composition may be administered in a single dose or in multiple doses. Administration of an amount that results in a maximum effect with a minimum amount without side effects is important in consideration of all of the above elements, and the amount may be easily determined by those skilled in the art.

In the method of preventing or treating Parkinson's disease, an administration route and method of the composition is not particularly limited, and any administration route and administration method may be followed as long as the composition including the composition may reach the target site. Specifically, the composition may be administered through various routes such as oral or parenteral, and non-limiting examples of the administration route include: oral, rectal, topical, intravenous, intraperitoneal, intramuscular, intraarterial, transdermal, and intranasal or inhalation routes.

Another aspect provides use of the composition including fusaric acid, ascorbic acid, nicotinamide adenine dinucleotide, or a combination thereof.

Another aspect provides use of a composition including dopaminergic neurons differentiated by the method of promoting differentiation of neural stem cells into dopaminergic neurons.

Another aspect provides subcultured neural stem cells used in the method of promoting differentiation of the subcultured neural stem cells into dopaminergic neurons.

Another aspect provides use of the composition for promoting differentiation of subcultured neural stem cells into dopaminergic neurons, incluing fusaric acid, ascorbic acid, nicotinamide adenine dinucleotide, or a combination thereof, for uses in a pharmaceutical composition or a formulation for preventing or treating Parkinson's disease.

Another aspect provides use of dopaminergic neurons prepared by the method of differentiating subcultured neural stem cells into dopaminergic neurons, for uses in preparation of a pharmaceutical composition or a formulation for preventing or treating Parkinson's disease.

Another aspect provides use of subcultured neural stem cells used in the method of differentiating subcultured neural stem cells into dopaminergic neurons, for uses in preparation of a pharmaceutical composition or a formulation for preventing or treating Parkinson's disease.

Another aspect provides use of the composition for promoting differentiation of subcultured neural stem cells into dopaminergic neurons, the composition incluing fusaric acid, ascorbic acid, nicotinamide adenine dinucleotide, or a combination thereof, for uses in preparation of a drug for preventing or treating a disease, for example, Parkinson's disease.

Another aspect provides use of dopaminergic neurons prepared by the method of differentiating subcultured neural stem cells into dopaminergic neurons, for uses in preparation of a drug for preventing or treating a disease, for example, Parkinson's disease.

Another aspect provides use of subcultured neural stem cells used in the method of differentiating subcultured neural stem cells into dopaminergic neurons, for uses in preparation of a drug for preventing or treating a disease, for example, Parkinson's disease.

The pharmaceutical composition, Parkinson's disease, prevention, and treatment are as described above.

### Advantageous Effects

A composition according to an aspect may increase differentiation of neural stem cells isolated from an early stage of fetal development into dopaminergic neurons, and may be commonly applicable to neural stem/progenitor cells isolated from fetuses of various weeks of age.

A composition according to another aspect may increase differentiation of neural stem cells subcultured 10 or more times into dopaminergic neurons, and thus, it is possible to secure more dopaminergic neurons, and increase therapeutic effects on Parkinson's disease.

### DESCRIPTION OF DRAWINGS

FIG. 1 is results of confirming expression of stem cell markers SOX2 and Nestin in neural stem cells isolated from the central nervous system of a 10-week-old fetus by immunostaining chemistry.

FIG. 2 is a diagram confirming the expression of TH, a marker of dopaminergic neurons, after differentiating FMD-NSPCs in a differentiation medium supplemented with FA alone, a combination of FA and AA, a combination of FA and NAD+, or a combination of FA, AA, and NAD+ (FA: 0.1 mM, AA: 0.2 μm, and NAD+: 1 mM).

FIG. 3 is a diagram confirming the expression of TH, a marker of dopaminergic neurons, and Tuj1, a marker of neural cells, after differentiating FMD-NSPCs in a differentiation medium supplemented with FA alone (FA: 0.1 mM).

FIG. 4 is a diagram confirming the expression of TH, a marker of dopaminergic neurons, and Tuj1, a marker of neural cells, after differentiating FMD-NSPCs obtained in subculture 9 in a differentiation medium supplemented with a combination of FA and AA (a), or a combination of FA and NAD+ (b).

FIG. 5 is a diagram confirming the expression of TH, a marker of dopaminergic neurons, after differentiating FMD-NSPCs obtained in subculture 12 in a differentiation medium supplemented with a combination of FA and AA (FA: 0.1 mM, AA: 0.2 mM).

FIG. 6 is a diagram confirming the expression of TH, a marker of dopaminergic neurons, after differentiating FMD-NSPCs obtained in subculture 14 (a) and subculture 15 (b) in a differentiation medium supplemented with a combination of FA and NAD+ (FA: 0.1 mM, NAD+ 0.5: 0.5 mM, NAD+ 1.0: 1 mM).

FIG. 7 is a diagram confirming the expression of TH, a marker of dopaminergic neurons, after differentiating FMD-NSPCs obtained in subculture 16 (a), subculture 17 (b), subculture 18 (c), and subculture 19 (d) in a differentiation medium supplemented with a combination of FA and AA (FA: 0.1 mM, AA: 0.2 mM).

FIG. 8 is a diagram confirming the expression of TH, a marker of dopaminergic neurons, after differentiating FMD-NSPCs obtained in subculture 21, a late stage, in a differentiation medium supplemented with a combination of FA and AA, or a combination of FA and NAD+.

FIG. 9 is a diagram confirming the expression of TH, a marker of dopaminergic neurons, and Tuj1, a marker of neural cells, after differentiating FMD-NSPCs obtained in subculture 10 (a), subculture 11 (b), and subculture 12 (c) in a differentiation medium supplemented with a combination of FA, AA, and NAD+ (FA: 0.1 mM, AA: 0.2 mM, NAD+: 1 mM).

FIG. 10 is a diagram confirming the expression of TH, a marker of dopaminergic neurons, and Tuj1, a marker of neural cells, after differentiating FMD-NSPCs obtained in subculture 17 and subculture 19 in a differentiation medium supplemented with FA alone, or a combination of FA, AA, and NAD+ (FA: 0.1 mM, AA: 0.2 mM, NAD+: 1 mM).

FIG. 11 is a diagram measuring fluorescence intensity by confirming the expression of TH, a marker of dopaminergic neurons, after differentiating FMD-NPCs in a differentiation medium supplemented with a combination of FA, AA, and NAD+.

### MODE FOR INVENTION

Hereinafter, the present disclosure will be described in more detail through examples. However, these examples are intended only to illustrate at least one embodiment, but not to limit the scope of the present disclosure thereto.

### Example 1. Isolation and Culture of Neural Stem Cells

Neural stem cells (FMD-NSPCs: fetal midbrain derived neural stem/progenitor cells) were isolated from the central nervous system of a 10-week-old fetus. Specifically, the human neural stem cells were isolated according to a method disclosed in Storch et al. 2001; Milosevic et al. in 2006, 2007 and the like. Ventral midbrain tissues from brain tissues of 10-week-old fetuses were isolated, and treated in a solution including 0.1 mg/ml of papain and 100 μg/ml of DNase at 37° C. for about 30 minutes, to separate into a single cell suspension. The suspension was washed with phosphate buffered saline (PBS) and incubated in 50 ug/ml of antipain at 37° C. for 30 minutes. The human neural stem cells (hNSPCs) obtained above were inoculated as a monolayer at a density of 30,000 cells/cm² on a culture dish coated with 15 μg/ml of poly-L-ornithine and 4 μg/ml of fibronectin, and cultured.

Thereafter, expression of SOX2 and Nestin was confirmed in the isolated human neural stem cells, by using immunostaining chemistry. Specifically, the isolated hNSPCs were washed three times with PBS and fixed with PBS containing 4% paraformaldehyde for 10 minutes. After washing three times with PBS, blocking was performed by reacting with PBS containing 3% normal goat serum, 0.2% Triton X-100, and 1% BSA at room temperature for one hour. Anti-nestin (rabbit anti-nestin, COVANCE, CA, USA) and anti-Sox2 (rabbit-anti-Sox2, Abcam) primary antibodies were incubated overnight, washed three times with PBS, and the obtained cells were incubated with secondary antibodies of anti-mouse (Alexa Fluor™ 488), anti-mouse (Alexa Fluor™ 594), anti-rabbit (Alexa Fluor™ 488), anti-rabbit (Alexa Fluor™ 594) antibodies at room temperature for 60 minutes, and stained (counterstained)with 4′-6-diamidino-2-phenylindole (DAPI).

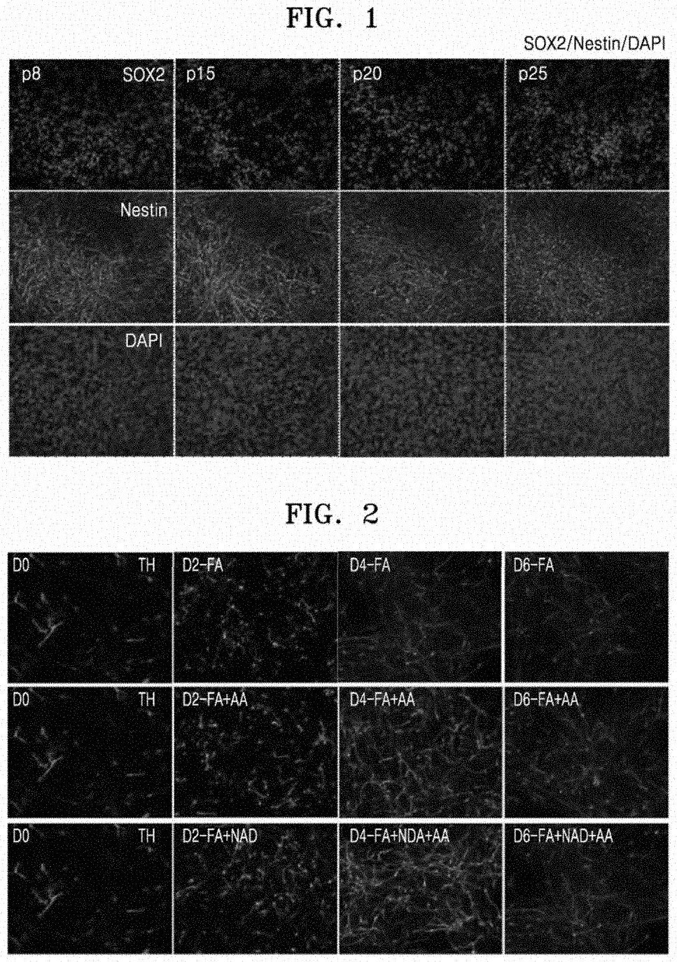

FIG. 1 is results of confirming expression of stem cell markers SOX2 and Nestin in neural stem cells isolated from the central nervous system of a 10-week-old fetus by immunostaining chemistry.

As a result, as shown in FIG.1, the isolated neural stem cells, unlike human neural progenitor cells (FMD-NPCs: fetal midbrain derived neural progenitor cells) isolated from a 14-week-old fetus, were confirmed to be cells obtained at an earlier stage than a developmental stage, through expression of stem cell markers SOX2 and Nestin.

### Example 2. Confirmation of Effects of Fusaric Acid, NAD+, and Ascorbic Acid of Enhancing Differentiation Potency of Neural Stem Cells into Dopaminergic Neurons

In order to confirm effects of NAD+, ascorbic acid (AA), and fusaric acid (FA) on differentiation potency of neural stem cells, human neural stem cells were differentiated in a differentiation medium (50 ml of Neurobasal Media ; B27-CTS or B27 supplement (50×) 1×, L-glutamine (100×) 1×, 10 μM of forskolin, 100 μM of dibutyryl cAMP) further supplemented with fusaric acid; fusaric acid and NAD+; fusaric acid and ascorbic acid; or fusaric acid, NAD+, and ascorbic acid, and differentiation potencies into dopaminergic neurons were compared (D2: day 2 of differentiation, D4: day 4 of differentiation, and D6: day 6 of differentiation).

Specifically, NSPCs derived from 14-week-old embryos and fetuses were cultured, allowed to settle, and differentiated for 6 days in a differentiation medium supplemented with FA alone, a combination of FA and AA, a combination of FA and NAD+, or a combination of FA, AA, and NAD+. The culture was continuously subcultured under a condition of oxygen partial pressure of 3% by changing the culture medium once every two days. The differentiated neural stem cells, that is, dopaminergic neurons were washed with a buffer solution after removing culture solution from the culture vessel, and treated with Accutase (PAA) for 30 minutes to separate the cells from the culture dish, and then again washed with a buffer solution. The obtained cells were centrifuged at 1,000 rpm for about 5 minutes to remove the supernatant, and the differentiated dopaminergic neurons were harvested. In order to compare differentiation potencies of neural stem cells into dopamine neurons, tyrosine hydroxylase (TH) antibodies (rabbit-anti-TH, Pelfreez), which is a marker of dopaminergic neurons, and Tuj1 antibodies (mouse anti-Tuj1 Millipore, CA. USA), a marker of neural cells were used to perform immunochemical staining.

As shown in FIG. 2, after differentiating FMD-NSPCs in a differentiation medium supplemented with FA alone, a combination of FA and AA, a combination of FA and NAD+, or a combination of FA, AA, and NAD+ (FA: 0.1 mM, AA: 0.2 μM, NAD+: 1 mM), expression of TH, a marker of dopaminergic neurons was confirmed, and as a result, differentiation into dopaminergic neurons was more clearly observed when a combination of FA and AA, a combination of FA and NAD+, or a combination of FA, AA, and NAD+ were added to the differentiation medium than when TH was added alone. In particular, when the neural cells were differentiated in a differentiation medium supplemented with a combination of FA, AA, and NAD+, differentiation into dopaminergic neurons was most prominently observed.

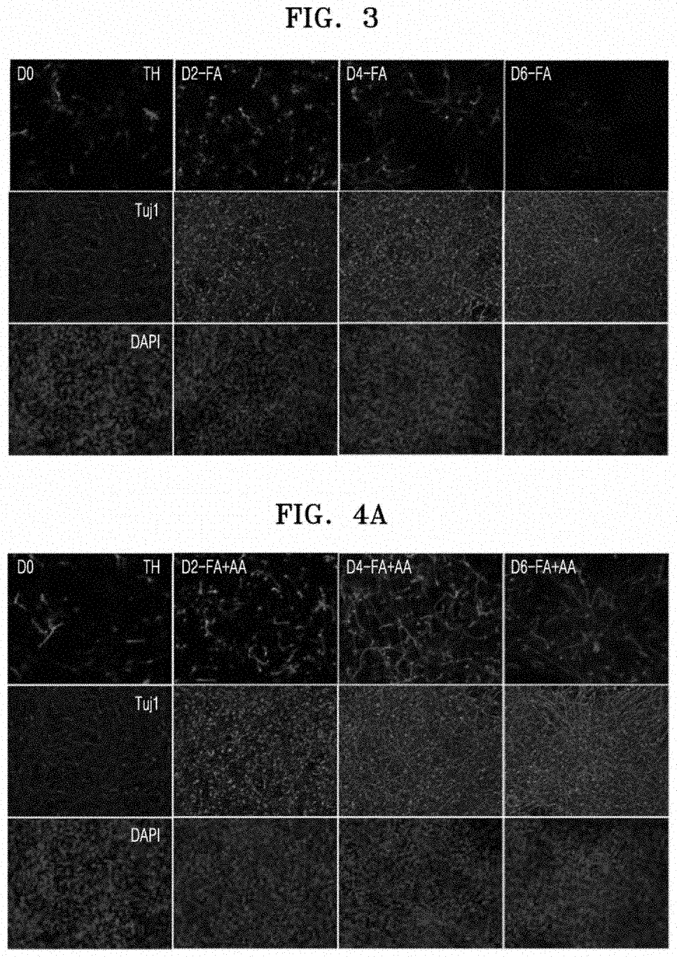

As shown in FIG. 3, after differentiating FMD-NSPCs in a differentiation medium (FA: 0.1 mM) supplemented with FA alone, expression of TH, a marker of dopaminergic neurons, and Tuj1, a marker of neural cells was confirmed, and as a result, as Tuj1 expression increased over time, stem cell characteristics decreased as differentiation progressed, and mature neurons increased due to gradual differentiation of proliferating cells. On the other hand, expression of TH decreased over time, confirming that differentiation into dopaminergic neurons was reduced.

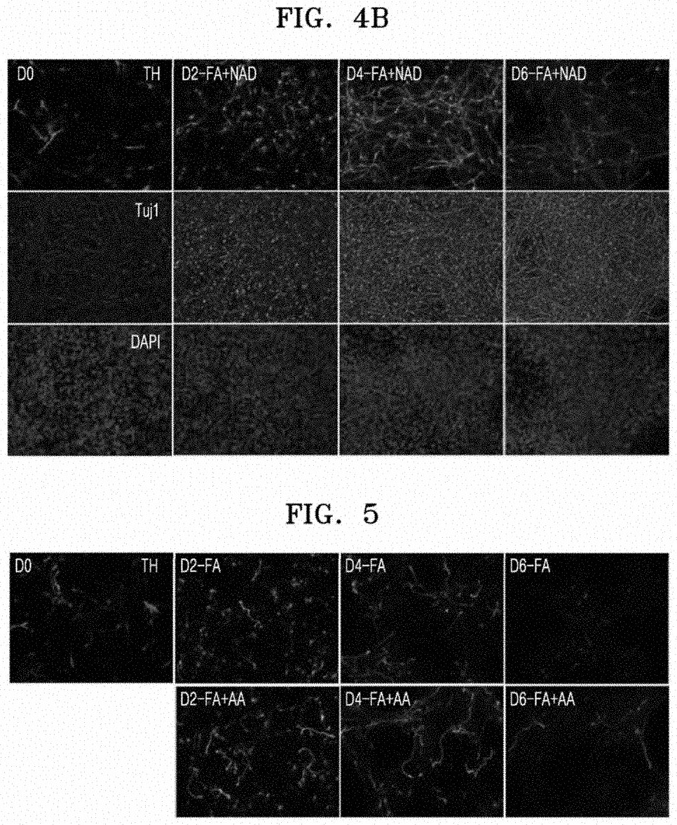

As shown in FIG. 4, after differentiating FMD-NSPCs obtained in subculture 9 in a differentiation medium supplemented with a combination of FA and AA (a), or a combination of FA and NAD+ (b) (FA: 0.1 mM, AA: 0.2 mM, and NAD+: 1 mM), expression of TH, a marker of dopaminergic neurons and Tuj1, a marker of neural cells was confirmed, and as a result of comparing with a case in which FA was added alone (compared with FIG. 3), it was confirmed that Tuj1 expression increased to a similar degree, but TH expression was maintained at a constant level without decreasing. That is, when neural stem cells were differentiated in a differentiation medium supplemented with a combination of FA and AA, or a combination of FA and NAD+, compared to a differentiation medium supplemented with FA alone, it was confirmed that differentiation of neural stem cells into dopaminergic neurons was increased.

As shown in FIG. 5, as a result of comparing expressions of TH, a marker of dopaminergic neurons, after differentiating FMD-NSPCs obtained in subculture 12 in a differentiation medium supplemented with FA alone or a combination of FA and AA, (FA: 0.1 mM, and AA: 0.2 mM), when the neural stem cells isolated from middle stage subcultures were differentiated in a medium supplemented with a combination of FA and AA, than in a medium supplemented with FA alone, differentiation efficiency of neural stem cells into dopaminergic neurons was confirmed to be slightly increased.

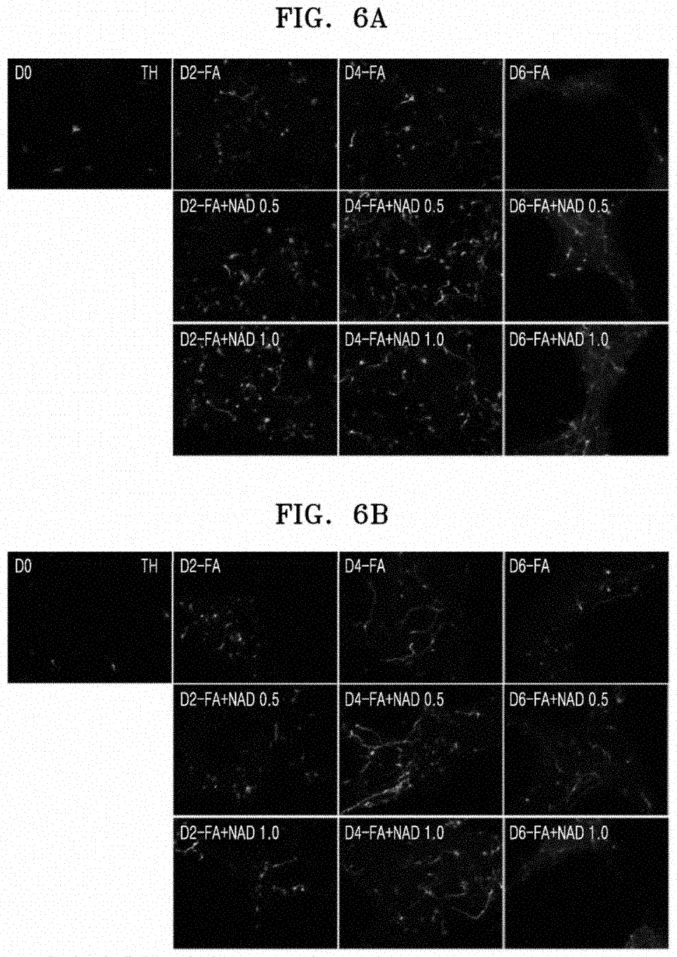

As shown in FIG. 6, as a result of comparing expressions of TH, a marker of dopaminergic neurons, after differentiating FMD-NSPCs obtained in subculture 14 (a) and subculture 15 (b) in a differentiation medium supplemented with FA alone or a combination of FA and NAD+ (FA: 0.1 mM, NAD+ 0.5: 0.5 mM, NAD+ 1.0: 1 mM), when the neural stem cells were differentiated in a medium supplemented with a combination of FA and NAD+, than in a medium supplemented with FA alone, differentiation efficiency of neural stem cells into dopaminergic neurons was confirmed to be slightly increased.

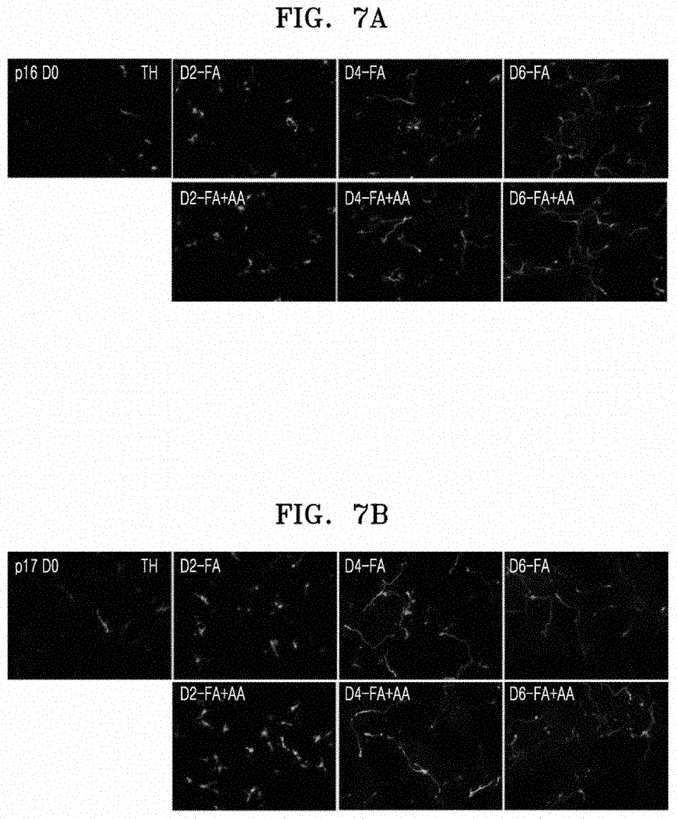

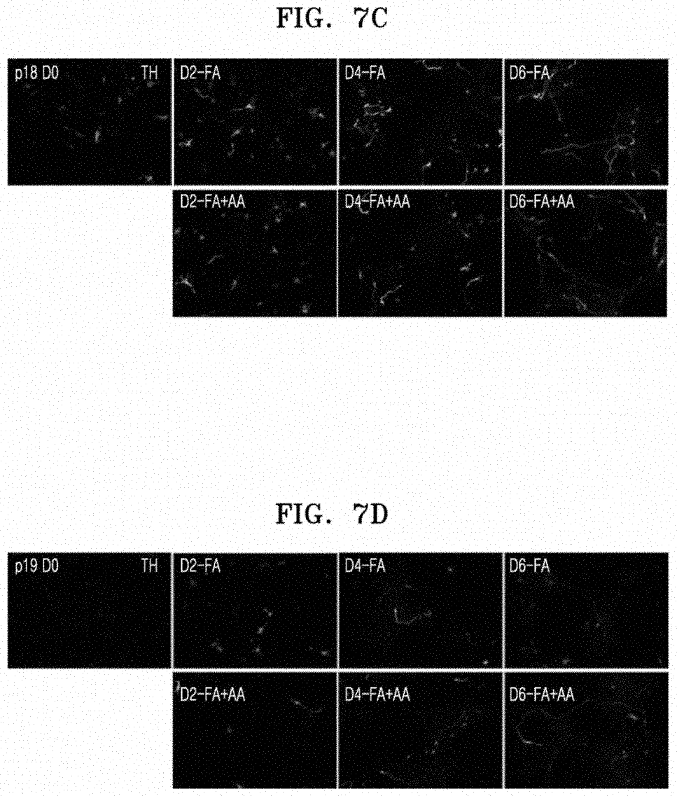

As shown in FIG. 7, as a result of comparing expressions of TH, a marker of dopaminergic neurons, after differentiating FMD-NSPCs obtained in subculture 16 (a), subculture 17 (b), subculture 18 (c), and subculture 19 (d) in a differentiation medium supplemented with FA alone, or a combination of FA and AA (FA: 0.1 mM, and AA: 0.2 mM), FMD-NSPCs isolated from late stage subcultures did not show significant differences in their ability to differentiate into dopaminergic neurons when differentiated in a differentiation medium supplemented with FA alone, or in a differentiation medium supplemented with a combination of FA and NAD+.

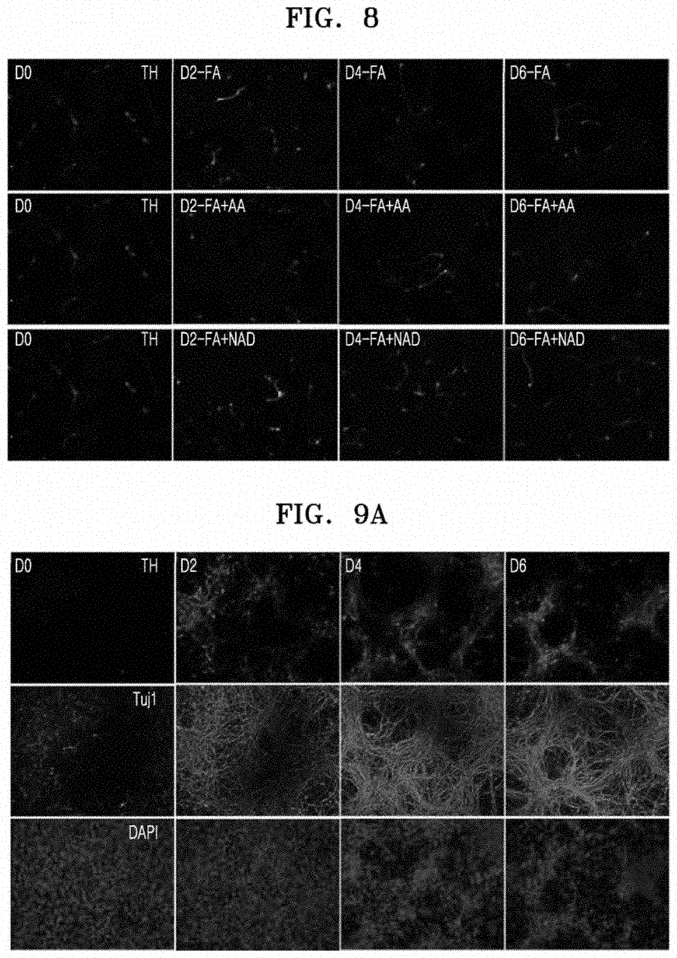

As shown in FIG. 8, as a result of comparing expressions of TH, a marker of dopaminergic neurons, after differentiating FMD-NSPCs obtained in subculture 21 in a differentiation medium supplemented with FA alone, a combination of FA and AA, or a combination of FA and NAD+, it was confirmed that even when AA or NAD+ was further added, respectively, than when FA was added alone, there was no significant effect on an ability of the neural stem cells isolated from a late stage subculture to differentiate into dopaminergic neurons.

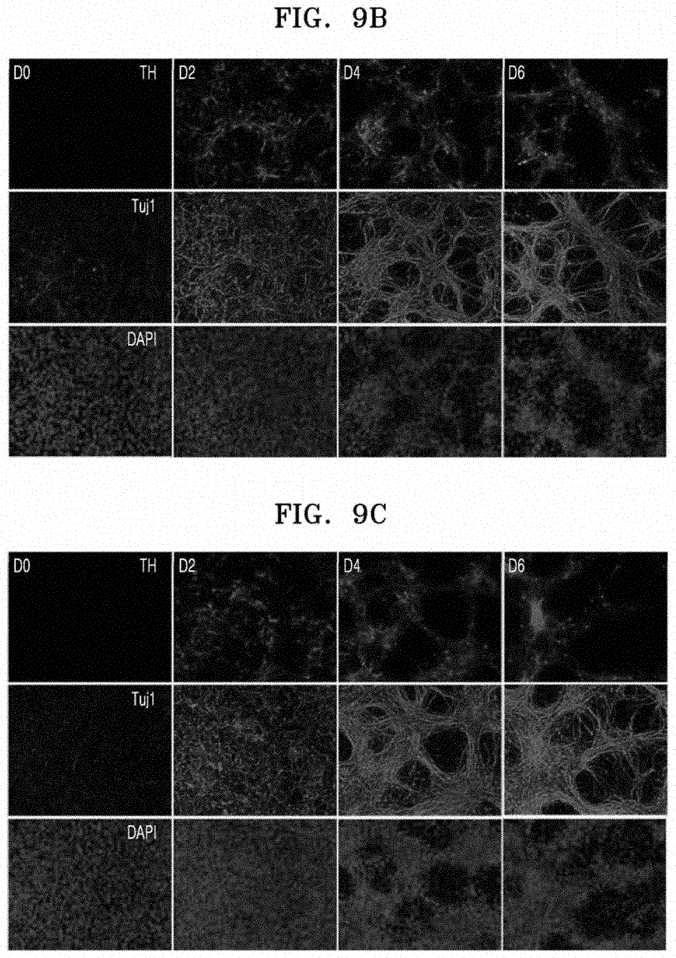

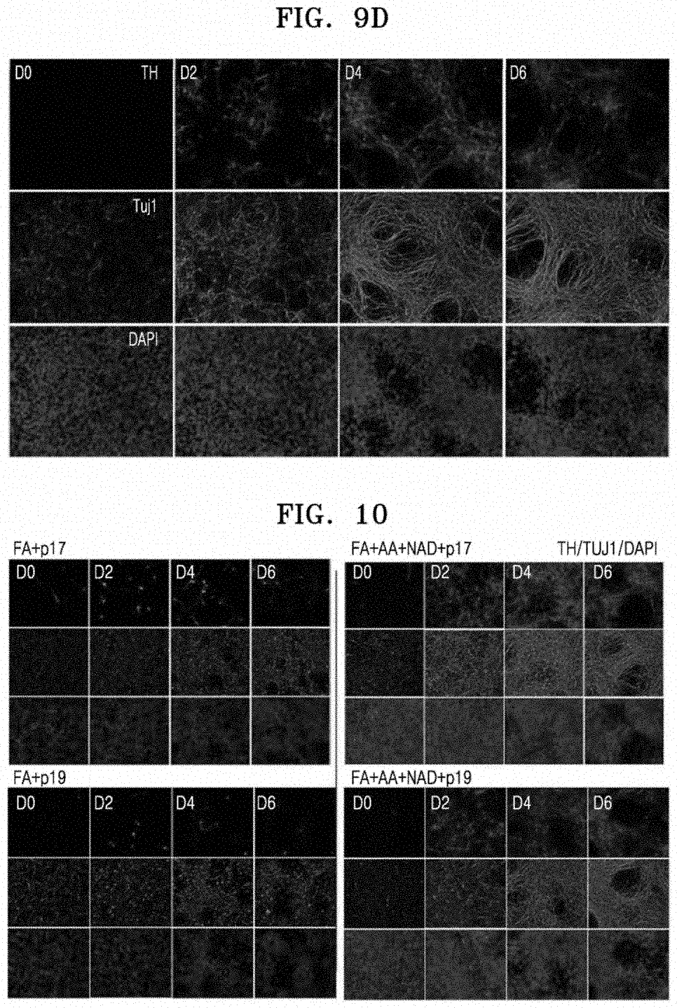

As shown in FIG. 9, as a result of comparing expressions of TH, a marker of dopaminergic neurons, and Tju1, a marker of neural stem cells, after differentiating FMD-NSPCs obtained in subculture 10 (a), subculture 11 (b), and subculture 12 (c), in a differentiation medium supplemented with a combination of FA, AA, and NAD+, it was confirmed that as Tuj1 expression increased, neural stem cells gradually differentiated and mature neurons increased, and since TH expression increased even until day 6 of differentiation, differentiation into dopaminergic neurons was increased.

As shown in FIG. 10, as a result of comparing expressions of TH, a marker of dopaminergic neurons, and Tju1, a marker of neural stem cells, after differentiating FMD-NSPCs obtained in subculture 17, and subculture 19, in a differentiation medium supplemented with FA alone, or a combination of FA, AA, and NAD+ (FA:0.1 mM, AA: 0.2 mM, and NAD+: 1 mM), even when differentiating neural stem cells isolated from late stage subcultures, when the neural stem cells were differentiated in a differentiation medium supplemented with FA, AA, and NAD+, than in a medium supplemented with FA alone, not only expression of TH was stronger, but also the differentiated cells showed a shape of a more mature neural cell in the development of the axon of a neural cell, and interconnection with the surrounding neural cells.

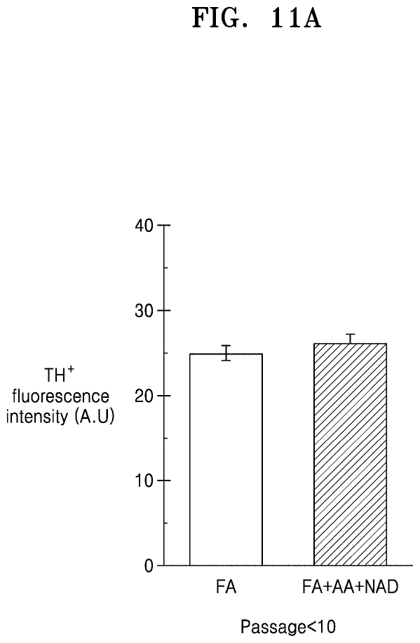

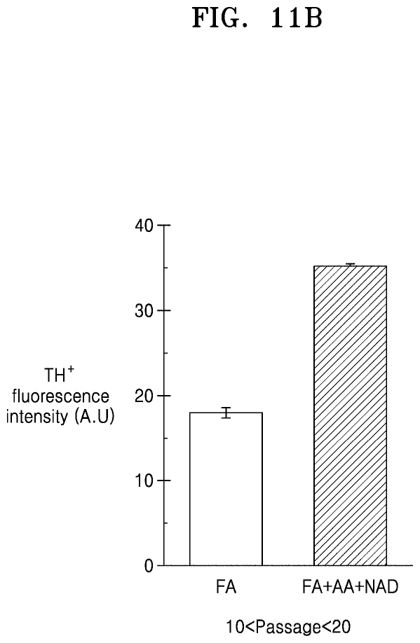

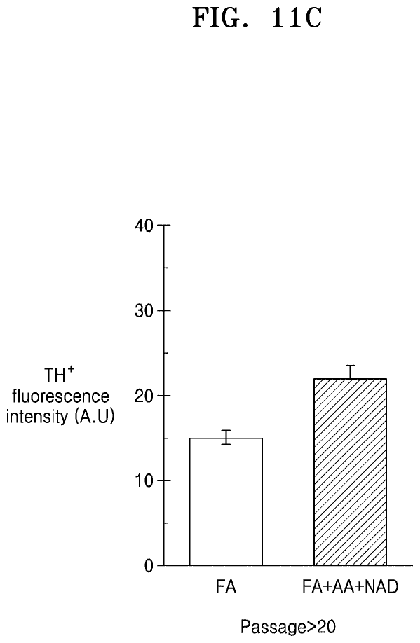

As shown in FIG. 11, after differentiating FMD-NPCs in a differentiation medium supplemented with FA alone, or a combination of FA, AA, and NAD+, expression of TH, a marker of dopaminergic neurons, was confirmed by measuring fluorescence intensity. For each group, neural stem cells (A) subcultured less than 10 times, (B) subcultured 10 or more times and 20 or less times, (C) subcultured 20 or more times were used to compare average values, to compare and analyze differentiation rates according to a subcultured number.

As a result, in cells treated with FA alone, a rate of differentiation into TH gradually decreased as the cells were subcultured.

In contrast, among the cells differentiated in the differentiation medium supplemented with a combination of FA, AA, and NAD+, there was no difference of differentiation rates in cells subcultured less than 10 times when using differentiation media supplemented with FA, and FA, AA, and NAD+.

In addition, as a result of differentiating cells subcultured 10 or more times and 20 or less times, it was confirmed that the differentiation rate was improved by about 2 times in the differentiation medium supplemented with FA, AA, and NAD+ than in the differentiation medium supplemented with FA alone, and about 1.3-fold improvement in the differentiation rate was confirmed in the cells subcultured 20 or more times.

From the above results, it was found that the differentiation of neural stem cells subcultured less than 10 times was less affected. In addition, neural stem cells subcultured 10 or more times and less than 20 times were most affected by the differentiation medium supplemented with a combination of FA, AA, and NAD+, and it was also confirmed that the differentiation rate increased in the cells subcultured 20 or more times in a differentiation medium supplemented with FA, AA, and NAD+. Therefore, it was confirmed that a differentiation rate of neural stem cells into dopaminergic cells, which gradually decreases when subcultured numbers increase, may be maintained or enhanced by culturing the neural stem cells in a differentiation medium supplemented with a combination of FA, AA, and NAD+.

Moreover, under the differentiation condition including a combination of FA, AA and NAD+, compared to the differentiation condition including FA alone, expression of NAD+ dependent histone deacetylases (SIRT1), which are known to inhibit intracellular protein entanglement and apoptosis caused by reactive oxygen stress, a pathogenesis of Parkinson's disease, may be maintained for a longer period of time. Therefore, it may be seen that the enhancement of an ability to differentiate into dopaminergic neurons by the combination of FA, AA, and NAD+ was achieved through increased SIRT1 activity.

1. A composition for promoting differentiation of neural stem cells into dopaminergic neurons, comprising fusaric acid, ascorbic acid, nicotinamide adenine dinucleotide (NAD+), or a combination thereof.

2. The composition of claim 1, comprising fusaric acid, ascorbic acid, and nicotinamide adenine dinucleotide.

3. The composition of claim 1, wherein the neural stem cells are subcultured 10 or more times and not more than 30 times.

4. The composition of claim 1, wherein the neural stem cells are subcultured 10 or more times and not more than 20 times.

5. The composition of claim 1, wherein the neural stem cells are embryonic stem cells, embryonic germ cells, embryonic carcinoma cells, induced pluripotent stem cells (iPSCs), or adult stem cells.

6. The composition of claim 1, wherein the dopaminergic neurons are dopaminergic neural progenitors, or dopaminergic neural precursor cells, or mature dopaminergic neurons.

7. The composition of claim 1, wherein the dopaminergic neurons are midbrain dopaminergic neurons.

8. A method of promoting differentiation of neural stem cells into dopaminergic cells, comprising: subculturing neural stem cells; and differentiating the subcultured neural stem cells in a medium comprising fusaric acid, ascorbic acid, nicotinamide adenine dinucleotide (NAD+), or a combination thereof.

9. The method of claim 8, wherein the subculturing is performed 10 or more times and not more than 30 times.

10. The method of claim 8, wherein the subculturing is performed 10 or more times and not more than 20 times.

11. The method of claim 8, wherein the medium comprises fusaric acid, ascorbic acid, and nicotinamide adenine dinucleotide.

12. Dopaminergic neurons differentiated by the method of claim 8.

13. A pharmaceutical composition for preventing or treating Parkinson's disease, comprising the dopaminergic neurons of claim 12 as active ingredients.

14. A method of preventing or treating Parkinson's disease, comprising:
administering the pharmaceutical composition of claim 13 to a subject.

15. A use of the composition of claim 1, for preparation of a drug for preventing or treating Parkinson's disease.
